(19) United States
(12) Patent Application Publication
(10) Pub. No.: US 2005/0147970 A1
(43) Pub. Date: Jul. 7, 2005

(54) Breast cancer associated polypeptide

(76) Inventors: Colin Copper, Surrey (GB); Jeremy Clark, Surrey (GB)

Correspondence Address: NIXON & VANDERHYE, PC, 1100 N GLEBE ROAD, 8TH FLOOR, ARLINGTON, VA, 22201-4714, US

(21) Appl. No.: 10/495,623

(22) PCT Filed: Nov. 14, 2002
(86) PCT No.: PCT/GB02/05166

(30) Foreign Application Priority Data
Nov. 14, 2001 (GB) 0127344.0

(51) Int. Cl. C12Q 1/68; C12P 19/34

(52) U.S. Cl. 435/6; 435/91.2

(57) ABSTRACT

This invention relates to the histone acetyl transferase protein HBO1 and its use in diagnosis and therapy of breast cancers. Methods and means relating to the diagnosis and treatment of breast cancer are provided.

### FIELD OF THE INVENTION

The present invention relates to the histone acetyl transferase protein HBO1 and its use in diagnosis and therapy of breast cancers.

### BACKGROUND TO THE INVENTION

Tumours arise through uncontrolled cell proliferation. Normally, cell proliferation is tightly regulated by a variety of mechanisms. These mechanisms rely, inter alia, on a large number of protein-protein interactions. Errors in these interactions, or the extent they take place, often underlie the start of the cancer process.

In many tumours, one underlying cause is the amplification and/or over-expression of genes resulting in aberrant expression of proteins. In some instances, large sections of a chromosome containing such genes are be amplified, resulting in the co-amplification of many other genes. A problem in dissecting the study of amplified regions is distinguishing between genes whose amplification (as well as, in some cases, over-expression) is a primary cause of tumour development and the co-amplification of genes which are in the same region of the chromosome as the amplified deleterious gene.

Gene amplification is frequently observed in human cancer and it is presumed that the over-expression of particular amplified genes confers selective advantage on individual clones of cancer cells by promoting growth and/or conferring drug resistance (Schwab & Aml, 1990; Schwab, 1998).

Amplifications often manifest cytogenetically as double minute chromosomes (DMs) and homogeneously staining regions (HSRs), and can be mapped within the genome by comparative genomic hybridisation (CGH) (Kallioniemi et al., 1992, 1993). Several strategies have been used to identify genes present within amplicons including “in gel” renaturation, positional cloning, hybrid selection of transcribed sequences from microdissected chromosomal regions and differential screening of cDNA libraries (Fukumoto et al., 1988; Tomasetto et al., 1995; Gracia et al., 1996; Collins et al., 1998). For breast cancer, conventional CGH onto metaphase chromosomes has determined that amplification events involve many distinct chromosomal regions, and molecular studies have identified candidate genes for many of the over represented regions. The regions most frequently amplified are at 17q11-21, 17q22-23 and 20q11-13.

Izuka and Stillman (1999) describe a novel protein termed HBO1, which has histone acetyl transferase activity. The mRNA encoding the protein was found to be expressed in a number of different human tissues, particularly ovarian tissue. HBO1 interacts with a protein, ORC1, which is a component of the human origin replication complex (ORC). The ORC is a key protein for the initiation of DNA replication in eukaryotes.

### DISCLOSURE OF THE INVENTION

Recent advances in microarray technology have provided a powerful means of identifying candidate oncogenes involved in DNA amplification events in tumours. However, microarray studies are limited by the number and choice of genes on an array. In order to gain a more complete view of gene amplification events, we have performed Comparative Genome Hybridisation (CGH) and expression analyses on microarrays of clones randomly selected from a cDNA library prepared from the cancer containing the DNA amplicon under investigation. When this approach was applied to the BT474 breast carcinoma cell line, which contains amplicons at 20q13, 17q11-21 and 17q22-23, we identified 50 amplified and expressed genes including many of the amplified genes previously proposed as having a role in breast cancer development. Microarray expression profiles together with analysis of the expression of amplified genes by Northern analyses in series of breast cancer cell lines identified a candidate oncogene, HB01, where gene amplification was consistently associated with higher levels of RNA expression. The results demonstrate the utility of this microarray-based CGH approach in hunting for candidate oncogenes within DNA amplicons.

The present invention thus provides a method of diagnosis or prognosis of breast cancer, which method comprises:
providing a sample of breast tissue which is, or is suspected to be, malignant from a patient;
determining the level of HBO1 expression or amplification in said sample; and
comparing said level with a control to determine whether the level of HBO1 expression is abnormal.

In another embodiment, the invention provides a method of identifying and/or obtaining an inhibitor of breast cancer cell growth, which method comprises:
providing HBO1 protein and a substrate therefor, under conditions where, in the absence of inhibitor, the protein is able to acetylate the substrate;
bringing a putative inhibitor into contact with said protein and substrate to determine if the putative inhibitor alters the ability of the HBO1 protein to acetylate the substrate; and
selecting a putative inhibitor which does alter the ability of the HBO1 protein to acetylate the substrate as an inhibitor of breast cancer cell growth.

An inhibitor obtained by this method forms a further aspect of the invention.

The invention further provides a method of inhibiting the growth of a breast cancer cell, which method comprises bringing the cell into contact with a histone acetyl transferase inhibitor capable of inhibiting HBO1 activity.

### DETAILED DESCRIPTION OF THE INVENTION

A method of assessing breast cancer as described above, may comprise:
determining the level of HBO1 expression or amplification in a sample of breast tissue obtained from a patient; and
comparing said level with a control to determine whether the level of HBO1 expression is abnormal.

Methods of assessing breast cancer may be useful in the diagnosis of individuals who have, or are suspected to have, a breast tumour. For example, the individual may be a female patient referred from a breast cancer screening programme for a follow up investigation.

In another aspect, the method may be used as a prognostic marker of clinical behaviour. For example, the subject may be one undergoing treatment for breast cancer and the screening will be used for monitoring the progression of the disease, the effectiveness of treatment—for example, the level of HB01 expression or amplification in breast tissue could be used to determine if the patient is responding to a particular course of treatment, and if not, the treatment course may be altered to try to obtain a more effective clinical outcome.

Levels of HBO1 expression may also me measured in secondary tumours in the patient, to monitor the spread of the tumour and the outcome of any clinical treatment against secondary tumours.

A sample suitable for use in the present methods may be obtained from a patient using routine techniques and may for example be a breast tissue biopsy.

Levels of HBO1 expression or amplification may be determined by analysis of DNA, RNA or of protein i.e. by measuring the amount of HBO1 nucleic acid or protein in a sample. The nucleotide and amino acid sequences of HBO1 may be obtained by reference to Izuka and Stillman (1999).

Various methods are available for determining the amount or level in a test sample of a particular nucleic acid sequence, for example an HBO1 RNA or cDNA.

To detect RNA (in the form of mRNA) nucleic acid probes to all or part of the gene may be made in accordance with routine procedures known as such in the art. Suitable methods include Southern blot analysis (for DNA copy number determination), fluorescent in-situ hybridization approaches, northern blotting or PCR-based approaches to determine mRNA copy number.

Methods may comprise determining, e.g. measuring, the level or amount of binding of an oligonucleotide probe to nucleic acid obtained from a sample, for example, RNA or cDNA. The probe may comprise a nucleotide sequence which binds specifically to a nucleotide sequence encoding all or part of the HBO1 polypeptide sequence.

The oligonucleotide probe may comprise a label and binding of the probe may be determined by detecting the presence of the label.

A method may include hybridisation of one or more (e.g. two) oligonucleotide probes or primers to target nucleic acid. The hybridisation may be as part of a PCR procedure, or as part of a probing procedure not involving PCR. An example procedure would be a combination of PCR and low stringency hybridisation.

Binding of a probe to target nucleic acid (e.g. DNA) may be measured using any of a variety of techniques at the disposal of those skilled in the art. For instance, probes may be radioactively, fluorescently or enzymatically labelled. Other methods not employing labelling of probe include examination of restriction fragment length polymorphisms and amplification using PCR. Probing may employ the standard Southern blotting technique. For instance, cDNA may be digested with different restriction enzymes. Restriction fragments may then be separated by electrophoresis on an agarose gel, before denaturation and transfer to a nitrocellulose filter. Labelled probe may be hybridised to the DNA fragments on the filter and binding determined.

Those skilled in the art are well able to employ suitable conditions of the desired stringency for selective hybridisation of probes, taking into account factors such as oligonucleotide length and base composition, temperature and so on. Suitable selective hybridisation conditions for oligonucleotides of 17 to 30 bases include hybridization overnight at 42° C. in 6×SSC and washing in 6×SSC at a series of increasing temperatures from 42° C. to 65° C.

Other suitable conditions and protocols are described in Molecular Cloning: a Laboratory Manual: 3rd edition, Sambrook & Russell, 2001, Cold Spring Harbor Laboratory Press NY and Current Protocols in Molecular Biology, Ausubel et al. eds., John Wiley & Sons, 1992.

HBO1 encoding nucleic acid molecules may be identified by specific hybridisation with a probe as described above or by sequencing. Sequencing of an amplified product may involve precipitation with isopropanol, resuspension and sequencing using a TaqFS+ Dye terminator sequencing kit. Extension products may be electrophoresed on an ABI 377 DNA sequencer and data analysed using Sequence Navigator software.

Since it will not generally be time- or labour-efficient to sequence all nucleic acid in a test sample or even the whole HBO1 coding sequence, a specific amplification reaction such as PCR using one or more pairs of primers may conveniently be employed to amplify a characteristic region of the HBO1 sequence. The level and/or amplification of HBO1 nucleic acid may be inferred from the presence or amount of amplified sequence produced. The amplified nucleic acid may be sequenced as above, if necessary, to confirm its identity.

Suitable amplification reactions include the polymerase chain reaction (PCR) (reviewed for instance in “PCR protocols; A Guide to Methods and Applications”, Eds. Innis et al, 1990, Academic Press, New York, Mullis et al, Cold Spring Harbor Symp. Quant. Biol., 51: 263, (1987), Ehrlich (ed), PCR technology, Stockton Press, NY, 1989, and Ehrlich et al, Science, 252: 1643-1650, (1991)). PCR comprises steps of denaturation of template nucleic acid (if double-stranded), annealing of primer to target, and polymerisation. The nucleic acid used as template in the amplification reaction may be DNA or, when used in combination with reverse transcriptase in an RT-PCR approach, RNA.

Other specific nucleic acid amplification techniques include strand displacement activation, the Qβ replicase system, the repair chain reaction, the ligase chain reaction, rolling circle amplification and ligation activated transcription. For convenience, and because it is generally preferred, the term PCR is used herein in contexts where other nucleic acid amplification techniques may be applied by those skilled in the art. Unless the context requires otherwise, reference to PCR should be taken to cover use of any suitable nucleic amplification reaction available in the art.

Methods of the present invention may therefore comprise amplifying all or part of the HBO1 coding sequence from one or more cells from said tissue sample. Preferably, the coding sequence is amplified from RNA within the one or more cells. for example by RT-PCR.

An oligonucleotide for use in nucleic acid amplification may be about 30 or fewer nucleotides in length (e.g. 18, 21 or 24). Generally specific primers are upwards of 14 nucleotides in length, but need not be than 18-20. Those skilled in the art are well versed in the design of primers for use processes such as PCR. Various techniques for synthesizing oligonucleotide primers are well known in the art, including phosphotriester and phosphodiester synthesis methods.

The level of HBO1 expression or amplification may be determined in a quantitative or qualitative manner. For example, in determining the amount of messenger RNA the copy number may be determined using an ABI PRISM 7700 sequence detection system and associated TaqMan probes. Such a system may also be used to determine levels of amplification by analysis of genomic DNA.

Hybridisation with HBO1 specific oligonucleotides may be conveniently carried out using an oligonucleotide array, preferably a microarray, to determine the presence or amount of HBO1 encoding nucleic acid in a sample, for example a sample of whole mRNA or cDNA from a cell (Yershov, G. et. al. (1996) PNAS USA, Genetics, Vol. 93, 4913-4918; Schena, M., 1999, DNA Microarrays “a practical approach”, ISBN, O-19-963777-6, Oxford press, editor B. D. Hames; Cheung, V. G., et. al., 1999, Nat. Genet., vol. 21, 15-19, WO84/01031, WO88/1058, WO89/01157, WO93/8472, WO95/18376/WO95/18377, WO95/24649 and EP-A-0373203).

In brief, the DNA microarray may be generated using oligonucleotides that have been selected to hybridise with the specific target sequence. These oligonucleotides may be applied by a robot onto a predetermined location of a glass slide, e.g. at predetermined X, Y cartesian coordinates, and immobilised. An nucleic acid sample (e.g. fluorescently labelled DNA) is introduced on to the DNA microarray and a hybridisation reaction conducted so that sample RNA or DNA binds to complementary oligonucleotide sequences in a sequence-specific manner, and unbound material is washed away. Target sequence is detected by its binding to complementary oligonucleotides on the array to produce a signal. The absence of a signal for a specific oligonucleotide probe indicates that the sample does not contain the corresponding sequence. The signal produced at each coordinate on the microarray is conveniently read using an automated detector in order to correlate each signal with a particular oligonucleotide.

Nucleic acid suitable for use in methods of the invention, such as an oligonucleotide probe and/or pair of amplification primers, may be provided as part of a kit, e.g. in a suitable container such as a vial in which the contents are protected from the external environment. The kit may include instructions for use of the nucleic acid, e.g. in PCR and/or a method for determining the presence of nucleic acid of interest in a test sample. A kit wherein the nucleic acid is intended for use in PCR may include one or more other reagents required for the reaction, such as polymerase, nucleosides, buffer solution etc. The nucleic acid may be labelled. A kit for use in determining the presence or absence of nucleic acid of interest may include one or more articles and/or reagents for performance of the method, such as means for providing or preparing the test sample itself.

Gene expression or amplification which is associated with breast cancer may also be detected at the protein level by measuring the level or amount of HBO1 polypeptide. An antibody against the HBO1 protein may, for example, be generated and used in an immunological assay in any suitable format. For practical purposes, or at least commercial purposes bearing in mind cost and time, assessment of target protein expression at the protein level may be preferred over assessment at the nucleic acid level.

An aspect of the invention provides a method of determining the presence or absence of cancer cells in a breast tissue sample obtained from an individual, the method including contacting a sample with a specific binding member directed against an HBO1 polypeptide, and determining the amount binding of the specific binding member to the sample, and comparing said level with a control to determine whether the level of HBO1 expression is abnormal.

Abnormal expression of HBO1 in a cell, for example over expression of HBO1, is indicative that the cell is a cancer cell.

As described above, binding of the specific binding member may be determined in a quantitative or qualitative manner. Binding of specific binding member to the sample may be compared to controls to determine the level of HBO1 expression or amplification. Increased HBO1 expression or amplification in a cell is indicative that the cell is a cancer cell, for example a breast cancer cell.

Another aspect of the present invention provides for a method of categorising a breast tissue sample as (i) normal or (ii) potentially or actually pre-cancerous or cancerous, dysplastic, or neoplastic, the method including determining the level of binding to a sample of the tissue of a specific binding member directed against an HBO1 polypeptide. The pattern or degree of binding may be compared with that for a known normal sample and/or a known abnormal sample.

Binding of (e.g.) an anti-HBO1 specific binding member to a sample provides for categorising the breast tissue from which the sample is derived as potentially or actually pre-cancerous or cancerous, dysplastic or neoplastic. The method may be used to pre-screen samples before further analysis. The method may be used for screening or analysis of samples previously tested using another technique.

A specific binding molecule may be provided in a kit, which may include instructions for use in accordance with a method of the invention. Such kits are provided as a further aspect of the invention. One or more other reagents may be included, such as labelling molecules, and so on (see below). Reagents may be provided within containers, which protect them from the external environment, such as a sealed vial. A kit may include one or more articles for providing or preparing the test sample itself depending on the tissue of interest. A kit may include any combination of, or all of, a blocking agent to decrease non-specific staining, a storage buffer for preserving binding molecule activity during storage, staining buffer and/or washing buffer to be used during antibody staining, a positive control, a negative control and so on. Positive and negative controls may be used to validate the activity and correct usage of reagents employed in accordance with the invention and which may be provided in a kit. Controls may include samples, such as tissue sections, cells fixed on cover-slips and so on, known to be either positive or negative for the presence of the HBO1. The design and use of controls is standard and well within the routine capabilities of those of ordinary skill in the art.

Samples to be subjected to contact with a binding member in accordance with methods of the invention may be prepared using any available technique which allows binding of a specific binding molecule to the HBO1 polypeptide. Various techniques are standard in the art.

The reactivities of a binding member such as an antibody on normal and test samples may be determined by any appropriate means. Tagging with individual reporter molecules is one possibility. The reporter molecules may directly or indirectly generate detectable, and preferably measurable, signals. The linkage of reporter molecules may be directly or indirectly, covalently, e.g. via a peptide bond or non-covalently. Linkage via a peptide bond may be as a result of recombinant expression of a gene fusion encoding binding molecule (e.g. antibody) and reporter molecule.

One favoured mode is by covalent linkage of a binding member with an individual fluorochrome, phosphor or laser dye with spectrally isolated absorption or emission characteristics. Suitable fluorochromes include fluorescein, rhodamine, phycoerythrin and Texas Red. Suitable chromogenic dyes include diaminobenzidine. Other reporters include macromolecular colloidal particles or particulate material such as latex beads that are coloured, magnetic or paramagnetic, and biologically or chemically active agents that can directly or indirectly cause detectable signals to be visually observed, electronically detected or otherwise recorded. These molecules may be enzymes that catalyse reactions that develop or change colours or cause changes in electrical properties, for example. They may be molecularly excitable, such that electronic transitions between energy states result in characteristic spectral absorptions or emissions. They may include chemical entities used in conjunction with biosensors. Biotin/avidin or biotin/streptavidin and alkaline phosphatase detection systems may be employed. Further examples are horseradish peroxidase and chemiluminescence.

The mode of determining binding is not a feature of the present invention and those skilled in the art are able to choose a suitable mode according to their preference and general knowledge.

Preferred specific binding molecules for use in aspects of the present invention include antibodies and fragments or derivatives thereof (‘antibody molecules’).

Antibodies that are specific for a HBO1 polypeptide may be obtained using techniques that are standard in the art. Methods of producing antibodies include immunising a mammal (e.g. mouse, rat, rabbit, horse, goat, sheep, monkey or bird such as chicken) with the HBO1 protein or a fragment thereof, or a cell or virus that expresses the protein or fragment.

Antibodies may be obtained from immunised animals using any of a variety of techniques known in the art, and screened, preferably using binding of antibody to mutant HBO1 polypeptide. For instance, Western blotting techniques or immunoprecipitation may be used (Armitage et al, 1992, Nature 357: 80-82). The production of specific monoclonal antibodies is also well established in the art.

As an alternative or supplement to immunising a mammal with a peptide, an antibody specific for a target may be obtained from a recombinantly produced library of expressed immunoglobulin variable domains, e.g. using lambda bacteriophage or filamentous bacteriophage which display functional immunoglobulin binding domains on their surfaces; for instance see WO92/01047. The library may be naive, that is constructed from sequences obtained from an organism that has not been immunised with the target or may be one constructed using sequences obtained from an organism that has been exposed to the antigen of interest (or a fragment thereof).

In methods of the invention, the level or expression or amplification in a tissue sample will be compared to a control. A control may be from a sample of tissue from the patient such as normal breast tissue or may be a historic control either from the patient or based on data from other samples or pools of sample. Where mRNA copy number is determined it may be suitable to use historic copy number data from the patient or patient populations that are desirably matched for factors such as age. Preferably the control is obtained from the same patient and the control may be repeated each time the sample is taken from the patient over the course of treatment of a disease or adjusting an initial biopsy which is used as a reference point for further determination of HBO1 expression in malignant tissue.

Where elevated HBO1 expression or amplification levels are determined this may be indicative of a malignant state, clinical behaviour or a response to drug treatment. The degree of elevated levels may also be used to monitor the progression of diseased state in a patient or to determine a course of therapeutic treatment.

The finding of elevated HBO1 levels in breast tumour cell lines is indicative that histone acetylation by this protein plays a role in the establishment and maintenance of disease. Therefore the protein and its acetylation activity is a novel target for the development of agents for the treatment of breast cancer. In a further aspect, the invention provides a method for identifying and/or obtaining an inhibitor of breast cancer cell growth, which method comprises providing HBO1 protein and a substrate therefore e.g. a histone, under conditions where, in the absence of inhibitor, the protein is able to acetylate the substrate;
bringing a putative inhibitor into contact with said protein and substrate to determine if the putative inhibitor alters the ability of the HBO1 protein to acetylate the substrate; and
selecting a putative inhibitor which does alter the ability of the HBO1 protein to acetylate the substrate as an inhibitor of breast cancer cell growth.

Histone acetylation may be conducted in the presence of a putative inhibitor compound and the degree to which the presence of this inhibitor reduces or prevents the acetylation of the histones occurring may then be determined. Inhibitors that show such activity may be selected as inhibitors of breast cancer cell growth.

A method may be configured in any suitable format for the determination of histone acetylation. Those of skill in the art may vary the precise format of the methods of the invention using routine skill and knowledge.

A method of identifying and/or obtaining a compound as a putative anti-cancer agent, in particular a putative anti-breast cancer agent, may comprise;
contacting an HBO1 polypeptide with a test compound and;
determining the acetylase activity of the HBO1 polypeptide.

The acetylase activity of the HBO1 polypeptide may be determined in the presence relative to the absence of test compound. A decrease in activity in the presence relative to the absence of test compound is indicative that the compound is a putative anti-cancer agent.

The acetylase activity of the HBO1 polypeptide may be determined as described below.

A compound, agent or inhibitor identified using one or more primary screens (e.g. in a cell-free system) as having ability to modulate the activity of HBO1 may be assessed further using one or more secondary screens. A secondary screen may involve testing for effects on the growth and/or proliferation of breast cancer cells in vitro or the growth or metastasis of breast cancer cells in an animal model.

A method may thus comprise the further step of; contacting in vitro a breast cancer cell with the test compound, and;
determining the growth of the cell.

To determine the acetylase activity of an HBO1 polypeptide, histone protein which may be in the form of free proteins or in a nuclear histone complex are provided together with HBO1 and an acetylation buffer. The acetylation buffer may include labelled acetyl co-enzyme A to allow the degree of acetylation to be determined. The HBO1 may be provided by immunoprecipitation of this protein from a cell extract as described in Izuka and Stillman (1999). This reference also describes in further detail various means by which histone acetylation can be determined.

Alternatively acetylation may be determined for example by immobilising histone protein, e.g. on a bead or plate, and detecting acetylation using an antibody or other binding molecule which binds the relevant site of acetylation with a different affinity when the site is acetylated from when the site is not acetylated. Such antibodies may be obtained by means of any standard technique as discussed elsewhere herein, e.g. using a acetylated histone peptide or fragment thereof. Binding of a binding molecule which discriminates between the acetylated and non-acetylated form of histone or relevant fragment thereof may be assessed using any technique available to those skilled in the art, which may involve determination of the presence of a suitable label.

Acetylation may also be assayed in solution, e.g. as described in Bannister and Kouzarides (1996), Nature, 384: 641-643. Briefly, protein substrate (˜1 μg) and ˜0.1 pmol of acetyl-transferase are mixed to give a final volume of 30 μl in buffer IPH (50 mM Tris.HCl pH8.0, 150 mM NaCl, 5 mM EDTA, 0.5% [v/v] NP-40, 0.1 mM PMSF). Reactions are initiated by the addition of [¹⁴C]-acetyl coA (1.85 kBq: 1.85 GBq/mmol; Amersham) and incubated at 30° C. for 10-45 min. The reaction products are then resolved by SDS-PAGE and viewed following fluorography of the gel. Alternatively, following SDS-PAGE, the resolved proteins can be Western blotted to a nitrocellulose membrane, which is then dried and exposed to film.

A further option is an in-gel activity assay, such as described by Brownell and Allis (1995), Proc. Natl. Acad. Sci., 92: 6364-6368 or Mizzen, et al (1996), Cell, 87: 1261-1270. Samples may be crude cellular extracts, partially purified fractions, highly purified cellular proteins or bacterially produced and purified recombinant proteins. Before loading onto the activity gel the sample is made to 1×SDS-PAG loading buffer and boiled for 2 minutes. The gel is a standard Laemmli SDS-PAG except that purified protein substrate is added to the resolving gel to a final concentration of 1 mg/ml. Polymerisation of the gel is initiated using standard techniques, at which point the protein substrate becomes immobilized within the gel matrix. After adding the stacking gel, the samples are loaded and the gel run as a standard SDS-PAG. After the gel has run it is subjected to various soakings before being exposed to [³H]-acetyl CoA is added for determination of activity.

The amount of test compound which may be employed in a method will normally be determined by trial and error depending upon the type of compound used. Typically, from about 0.01 to 100 nM concentrations of putative modulator compound may be used, for example from 0.1 to 10 nM. Modulator compounds may be those which either agonise or antagonise the interaction. Antagonists (inhibitors) of the interaction are particularly desirable.

Test compounds which may be used include natural or synthetic chemical compounds used in drug screening programmes. Extracts of plants or microorganisms which contain several characterised or uncharacterised components may also be used.

One class of putative inhibitor compounds can be derived from the HBO1 polypeptide sequence. Membrane permeable peptide fragments of from 5 to 40 amino acids, for example, from 6 to 10 amino acids may be tested for their ability to disrupt such interaction or activity.

A convenient way of producing polypeptide molecules, which may full-length sequences or peptide fragments thereof, for use in methods of the invention, is to express nucleic acid encoding it, by use of nucleic acid in an expression system.

Peptide fragments may also be generated wholly or partly by chemical synthesis. The compounds of the present invention can be readily prepared according to well-established, standard liquid or, preferably, solid-phase peptide synthesis methods, general descriptions of which are broadly available (see, for example, in J. M. Stewart and J. D. Young, Solid Phase Peptide Synthesis, 2nd edition, Pierce Chemical Company, Rockford, Ill. (1984), in M. Bodanzsky and A. Bodanzsky, The Practice of Peptide Synthesis, Springer Verlag, New York (1984); and Applied Biosystems 430A Users Manual, ABI Inc., Foster City, Calif.), or they may be prepared in solution, by the liquid phase method or by any combination of solid-phase, liquid phase and solution chemistry, e.g. by first completing the respective peptide portion and then, if desired and appropriate, after removal of any protecting groups being present, by introduction of the residue X by reaction of the respective carbonic or sulfonic acid or a reactive derivative thereof.

The inhibitory properties of a peptide fragment as described above may be increased by the addition of one of the following groups to the C terminal: chloromethyl ketone, aldehyde and boronic acid. These groups are transition state analogues for serine, cysteine and threonine proteases. The N terminus of a peptide fragment may be blocked with carbobenzyl to inhibit aminopeptidases and improve stability (Proteolytic Enzymes 2nd Ed, Edited by R. Beynon and J. Bond, Oxford University Press, 2001).

Antibodies directed to the active site of the HBO1 polypeptide form a further class of putative inhibitor compounds. Candidate inhibitor antibodies may be characterised and their binding regions determined to provide single chain antibodies and fragments thereof which disrupt the interaction.

Techniques for obtaining antibodies are standard in the art and are described elsewhere herein.

Antibody molecules may for example be micro-injected into cells, e.g. at a tumour site, subject to radio- and/or chemotherapy. Antibodies may be employed in accordance with the present invention for other therapeutic and non-therapeutic purposes, which are discussed elsewhere herein.

Other candidate inhibitor compounds may be based on modelling the 3-dimensional structure of a polypeptide or peptide fragment and using rational drug design to provide potential modulator (for example, inhibitor) compounds with particular molecular shape, size and charge characteristics.

A potential modulator compound may be a “functional analogue” of a peptide or other compound that modulates HBO1 activity. A functional analogue has the same functional activity as the peptide or other compound in question, i.e. it may interfere with the binding between HBO1 and one or more ligands, and or inhibit acetylation. Examples of such analogues include chemical compounds which are modelled to resemble the three dimensional structure of the HBO1 polypeptide in the contact area, and in particular the arrangement of the key amino acid residues as they appear in the form of HBO1.

HBO1 polypeptide and its binding partners may be used in methods of designing mimetics of these molecules suitable for inhibiting HBO1 activity.

Accordingly, the present invention provides a method of designing mimetics of HBO1 polypeptide having the biological activity of activity of modulating, e.g. inhibiting, HBO1 acetylase activity, said method comprising:
(i) analysing a substance having the biological activity to determine the amino acid residues essential and important for the activity to define a pharmacophore; and,
(ii) modelling the pharmacophore to design and/or screen candidate mimetics having the biological activity.

Suitable modelling techniques are known in the art. This includes the design of so-called “mimetics” which involves the study of the functional interactions of the molecules and the design of compounds which contain functional groups arranged in such a manner that they could reproduced those interactions.

The modelling and modification of a ‘lead’ compound to optimise its properties, including the production of mimetics, is further described below.

As described above, the activity or function of HBO1 may be inhibited, as noted, by means of a compound that interferes in some way with the interaction of HBO1 with other factors described herein. An alternative approach to inhibition employs regulation at the nucleic acid level to inhibit activity or function by down-regulating production of HBO1. For instance, expression of a gene may be inhibited using anti-sense or RNAi technology, as described elsewhere herein.

Methods of the present invention may include identifying the test compound as a putative anti-cancer agent, in particular, an anti-breast cancer agent. Methods may further include isolating, purifying, synthesising and/or manufacturing a compound identified as a putative anti-cancer agent.

Optionally, compounds identified as putative anti-cancer agents using a method described herein may be modified to optimise activity or provide other beneficial characteristics such as increased half-life or reduced side effects upon administration to an individual.

Methods of the present invention may further include formulating a compound identified as a putative anti-cancer agent into a composition, such as a medicament, pharmaceutical composition or drug, with a pharmaceutically acceptable excipient as described below.

Another aspect of the invention provides a pharmaceutical composition comprising the putative anti-cancer agent as described above. Such compositions will comprise the compound together with suitable carriers, diluents and excipients. Such formulations form a further aspect of the present invention and may be administered to an individual in the treatment of breast cancer. Compositions may additionally contain a second agent for the treatment of breast cancer, such as anti-hormonal agents such as tamoxifen; 5-fluorouracil; anthracylines such as doxorubicin (adriamycin); cyclophophamide; aromatase inhibitors; taxanes; or herceptin.

The inhibitor compounds may also be used in conjunction with other therapeutic approaches, for example radiotherapy, gene therapy or immunotherapy.

A method of making a pharmaceutical composition may comprise,
identifying a compound as inhibitor of HBO1 acetylase activity using a method described herein,
synthesising, preparing or isolating said inhibitor and
admixing the inhibitor with a pharmaceutically acceptable excipient, vehicle or carrier, and optionally other ingredients to formulate or produce said composition; and, optionally,
determining the acetylase activity of HBO1 as described herein in the presence of said composition.

In other embodiments, a method of producing a pharmaceutical composition may comprise;
identifying a compound which modulates the activity of an HBO1 polypeptide using a method described herein; and,
admixing the compound identified thereby with a pharmaceutically acceptable carrier.

The formulation of compositions with pharmaceutically acceptable carriers is described further below.

Another aspect of the invention provides a method for preparing a pharmaceutical composition, for example, for the treatment of breast cancer comprising;
i) identifying a compound which is an antagonist of a HBO1 polypeptide
ii) synthesising the identified compound, and;
iii) incorporating the compound into a pharmaceutical composition for use in the treatment of breast cancer.

Such a composition may comprise one or more other agents useful in the treatment of breast cancer as described above.

The identified compound may be synthesised using conventional chemical synthesis methodologies. Methods for the development and optimisation of synthetic routes are well known to a skilled person.

The compound may be modified and/or optimised as described above.

Incorporating the compound into a pharmaceutical composition may include admixing the synthesised compound with a pharmaceutically acceptable carrier or excipient.

The modification of a known pharmacologically active compound to improve its pharmaceutical properties is a known approach to the development of pharmaceuticals based on a “lead” compound. This might be desirable where the active compound is difficult or expensive to synthesise or where it is unsuitable for a particular method of administration, e.g. peptides are not well suited as active agents for oral compositions as they tend to be quickly degraded by proteases in the alimentary canal. The design, synthesis and testing of modified active compounds, including mimetics, may be used to avoid randomly screening large number of molecules for a target property.

There are several steps commonly taken in modifying a compound which has a given target property. Firstly, the particular parts of the compound that are critical and/or important in determining the target property are determined. In the case of a peptide, this can be done by systematically varying the amino acid residues in the peptide, e.g. by substituting each residue in turn. These parts or residues constituting the active region of the compound are known as its “pharmacophore”.

Once the pharmacophore has been found, its structure is modelled to according its physical properties, e.g. stereochemistry, bonding, size and/or charge, using data from a range of sources, e.g. spectroscopic techniques, X-ray diffraction data and NMR. Computational analysis, similarity mapping (which models the charge and/or volume of a pharmacophore, rather than the bonding between atoms) and other techniques can be used in this modelling process.

In a variant of this approach, the three-dimensional structure of the mutant HBO1 and ligands are modelled. This can be especially useful where the ligand and/or binding partner change conformation on binding, allowing the model to take account of this the design of the mimetic.

A template molecule is then selected onto which chemical groups which mimic the pharmacophore can be grafted. The template molecule and the chemical groups grafted on to it can conveniently be selected so that the modified compound is easy to synthesise, is likely to be pharmacologically acceptable, and does not degrade in vivo, while retaining the biological activity of the lead compound. Modified compounds found by this approach can then be screened to see whether they have the target property, or to what extent they exhibit it. Further optimisation or modification can then be carried out to arrive at one or more final compounds for in vivo or clinical testing.

Those of skill in the art may vary the precise format of methods of the invention using routine skill and knowledge.

Another aspect of the invention provides a method of treating a cancer condition, such as breast cancer, in an individual, the method comprising inhibiting the histone acetylase activity of HBO1 polypeptide in one or more cells of said individual.

The histone acetylase activity of HBO1 polypeptide may be inhibited, for example, by administering an antagonist of HBO1 to said individual. A suitable antagonist may be an antibody molecule, peptide or small organic molecule, and may be obtained by a method described above.

A method for inhibiting the growth of a breast cancer cell may for example, comprise bringing the cell in to contact with a histone acetyl transferase inhibitor capable of inhibiting HBO1 activity.

An alternative approach to inhibition employs regulation at the nucleic acid level to inhibit activity or function by down-regulating production of HBO1. The activity of HBO1 polypeptide may be inhibited by reducing or abolishing expression of the HBO1 polypeptide using anti-sense or RNAi technology. The use of these approaches to down-regulate gene expression is now well-established in the art.

Anti-sense oligonucleotides may be designed to hybridise to the complementary sequence of nucleic acid, pre-mRNA or mature mRNA, interfering with the production of HBO1 polypeptide so that its expression is reduced or completely or substantially completely prevented. In addition to targeting coding sequence, anti-sense techniques may be used to target control sequences of a gene, e.g. in the 5′ flanking sequence, whereby the antisense oligonucleotides can interfere with expression control sequences. The construction of antisense sequences and their use is described for example in Peyman and Ulman, Chemical Reviews, 90: 543-584, (1990) and Crooke, Ann. Rev. Pharmacol. Toxicol., 32: 329-376, (1992).

Oligonucleotides may be generated in vitro or ex vivo for administration or anti-sense RNA may be generated in vivo within cells in which down-regulation is desired. Thus, double-stranded DNA may be placed under the control of a promoter in a “reverse orientation” such that transcription of the anti-sense strand of the DNA yields RNA which is complementary to normal mRNA transcribed from the sense strand of the target gene. The complementary anti-sense RNA sequence is thought then to bind with mRNA to form a duplex, inhibiting translation of the endogenous mRNA from the target gene into protein. Whether or not this is the actual mode of action is still uncertain. However, it is established fact that the technique works.

The complete sequence corresponding to the coding sequence in reverse orientation need not be used. For example fragments of sufficient length may be used. It is a routine matter for the person skilled in the art to screen fragments of various sizes and from various parts of the coding or flanking sequences of a gene to optimise the level of anti-sense inhibition. It may be advantageous to include the initiating methionine ATG codon, and perhaps one or more nucleotides upstream of the initiating codon. A suitable fragment may have about 14-23 nucleotides, e.g. about 15, 16 or 17.

An alternative to anti-sense is to use a copy of all or part of the target gene inserted in sense, that is the same, orientation as the target gene, to achieve reduction in expression of the target gene by co-suppression; Angell & Baulcombe (1997) The EMBO Journal 16, 12: 3675-3684; and Voinnet & Baulcombe (1997) Nature 389: pg 553). Double stranded RNA (dsRNA) has been found to be even more effective in gene silencing than both sense or antisense strands alone (Fire A. et al Nature, Vol 391, (1998)). dsRNA mediated silencing is gene specific and is often termed RNA interference (RNAi).

RNA interference is a two-step process. First, dsRNA is cleaved within the cell to yield short interfering RNAs (siRNAs) of about 21-23 nt length with 5′ terminal phosphate and 3′ short overhangs (˜2 nt). The siRNAs target the corresponding mRNA sequence specifically for destruction (Zamore P. D. Nature Structural Biology, 8, 9, 746-750, (2001).

RNAi may be also be efficiently induced using chemically synthesized siRNA duplexes of the same structure with 3′-overhang ends (Zamore PD et al. Cell, 101, 25-33, (2000)). Synthetic siRNA duplexes have been shown to specifically suppress expression of endogenous and heterologeous genes in a wide range of mammalian cell lines (Elbashir S M. et al. Nature, 411, 494-498, (2001)).

Another possibility is that nucleic acid is used which on transcription produces a ribozyme, able to cut nucleic acid at a specific site—thus also useful in influencing gene expression. Background references for ribozyrnes include Kashani-Sabet and Scanlon, 1995, Cancer Gene Therapy, 2(3): 213-223, and Mercola and Cohen, 1995, Cancer Gene Therapy, 2(1), 47-59.

Thus, an inhibitor of HBO1 activity may comprise a nucleic acid molecule comprising all or part of the HBO1 coding sequence or the complement thereof.

Such a molecule may suppress the expression of the HBO1 polypeptide and may comprise a sense or anti-sense HBO1 coding sequence or may be a HBO1 specific ribozyme, according to the type of suppression to be employed.

The type of suppression will also determine whether the molecule is double or single stranded and whether it is RNA or DNA. Examples of the use of siRNA to reduce or abolish HBO1 expression are provided below.

Further aspects of the invention provide a nucleic acid encoding HBO1 polypeptide or its complement or a fragment thereof for use in a method of treatment, for example in a method of treating a cancer condition such as breast cancer and the use of nucleic acid encoding HBO1 or its complement or a fragment thereof in the manufacture of a medicament for the treatment of cancer, in particular breast cancer.

Whether it is a polypeptide, peptide, nucleic acid molecule, small molecule or other pharmaceutically useful compound that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors.

A composition may be administered alone or in combination with other treatments, either simultaneously or sequentially dependent upon the condition to be treated.

Pharmaceutical compositions according to the present invention, and for use in accordance with the present invention, may include, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous or intravenous.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, or Lactated Ringer's Injection. Preservatives, stabilisers, buffers, antioxidants and/or other additives may be included, as required.

Aspects of the present invention will now be illustrated with reference to the following experimental exemplification, by way of example and not limitation. Further aspects and embodiments will be apparent to those of ordinary skill in the art. All documents mentioned in this specification are hereby incorporated herein by reference.

Analysis of breast cancer amplicons at 17q has revealed independently amplified regions at 17q11-21 and 17q22-23. The PS6K serine-threonine kinase gene and the SIGMAIB (AP-2 clathrin adapter protein complex small subunit) gene have been proposed as candidates for involvement in the amplicon at 17q22-23 which has been found in up to. 28% of primary breast cancers (Couch et al., 1999; Wu et al., 2000).

Recently, Pollack et al. (1999) have identified new genes present within the 17q11-21, 17q22-23 and 20q13 breast cancer amplicons by carrying out CGH directly onto microarrays of a set of 5000 cDNA clones and ESTs. Interestingly they found that the clones showing the highest level of amplification were not previously characterised candidate oncogenes but anonymous ESTs, suggesting that the relevant oncogenes in these regions may remain uncharacterised. The disadvantage of their approach is that it can only investigate genes preselected for inclusion on the microarray. To overcome this limitation in the current study, we have performed CGH on arrays of clones randomly selected from a cDNA library prepared from a breast cancer cell line containing the 20q13, 17q11-21 and 17q22-23 amplicons. To facilitate identification of candidate oncogenes where amplification is accompanied by increased expression, we have in parallel studies (i) hybridised the microarrays to cDNA targets prepared from breast cancer cell lines, and (ii) carried out Northern analyses on selected genes.

Materials and Methods

Cell Lines

Nine established breast cell lines BT474, BT549, DU4475, MCF7, MDA-MB-134, MDA-MB-157, MDA-MB-361, SK-BR-3, ZR-75-1, were obtained from the American Type Culture Collection. Cells were grown according to the supplier's instructions. The conditionally immortalised human mammary luminal epithelial cell line HB4a was kindly donated by M J O'Hare. Since reports of amplicons on chromosomes 20 and 17 in the literature are not consistent, we used Southern blot analysis to re-examine the cell lines for the presence of amplicons at 20q13, 17q11-21 and 17q22-23. We established that the BT474 and MDA-MB-361 cell lines contains all three amplicons and that the SK-BR-3 cell line contains amplicons at only 20q13 and 17q11-21 (see Table 1). The MCF7 cell line contained an amplicon only at 20q13. We failed to detect amplification of these regions in the remaining cell lines.

DNA and RNA Extraction and Analyses

Genomic DNA was prepared using established methods (Sambrook et al., 1989). RNA was harvested from cytoplasmic cell lysates as described previously (Wilkinson, 1988), or using TRIZOL reagent (Gibco-BRL). Southern and Northern analyses were carried out as Sambrook et al., (1989). Equal sample loading in the gels was assessed by ethidium bromide staining and UV transillumination.

Probes for Southern and Northern blots were usually PCR amplified from cDNA clones identified on microarrays, and labelled with [α-³²P]-dCTP using a Bioprime kit (Life Technologies) according to the manufacturers instructions. DNA sequencing of PCR products was performed using a BigDye Terminator Cycle Sequencing kit (Perkin-Elmer) using the manufacturers protocols.

Microarray Libraries

A cDNA library from the BT474 breast carcinoma cell line was made using a cDNA synthesis kit (Stratagene) as described previously (Clark et al., 1994) except that the cDNA was ligated into the EcoRI/XhoI sites of pBluescript (Stratagene) and transformed into XL10-Gold Ultracompetent bacteria (Stratagene). Library cDNA clones were picked into 96 well plates using a colony picker robot (Flexys) and grown overnight in L-broth with 7% (v/v) glycerol and ampicillin selection. 1 μl of each culture was used to seed an 80 μl PCR mix set up in a 96-well plate. PCR mixes contained ABGene (Epsom) Buffer IV, 1-5 mM MgCl₂, 0-1 μM primers (M−13F GTAAAACGACGGCCAGTG and M-13R GGAAACAGCTATGACCATG) and 1-6 units of Thermoprime-Plus DNA polymerase (ABGene, Epsom). The PCR cycle consisted of 1×95° C. for 5 min, then 30 rounds of: 95° C., 1 min; 60° C., 1 min; 68° C., 3 min. PCR products were harvested by adding NH₃Ac to 2-5M, 0-6 volumes of propan-2-ol and centrifugation at 2,000 g for 2 hr. Pellets were washed with 80% (v/v) ethanol and redissolved in 10 μl H₂O o/n 4° C. 10 μl DMSO was added, mixed, and the products transferred to 384 well plates for gridding. cDNA insert sizes varied from 0-5 to 3 kb.

The “ICR-geneset” microarray consisted of 5,603 IMAGE cDNA clones acquired from the Human Genome Mapping Project Resource Centre (http://www.hgmp.mrc.ac.uk) and from Research Genetics (http://www.resgen.com). Clones with defined biological functions were selected in preference to anonymous ESTs (5,321 known function:282 anonymous ESTs). Information on the ICR-geneset can be obtained at http://icr.ac.uk/array/array.html. cDNA inserts were prepared for gridding as described above except that the clones were amplified with vector primers GAGCGGATAACAATTTCACACAG and GTTTTCCCAGTCACGACG, in buffer containing 6-7 mM MgCl₂, 66 mM Tris Base pH 8-8, 16-6 mM (NH₄)₂SO₄, 5 mM P-mercaptoethanol, 10 μM EDTA.

Microarray Slides

Gold Seal glass slides (BDH) were first rinsed in 1 M NaOH in 57% (v/v) ethanol for 2 hr and washed thoroughly with water. Slides were agitated in 11% (w/v) poly-L-lysine (Sigma), 0-1×PBS for 1 hr and then rinsed in water. This poly-L-lysine treatment was repeated. The slides were gently centrifuged to remove excess water and then air-dried. PCR products were applied in an 11×11×48 format to poly-L-lysine coated glass slides using a Flexys Genomic Solution robot fitted with a solid pin arrayer. Grid spots were 400 μm apart centre to centre and approximately 250 μm in diameter. The slides were exposed to 65K μJ UV light in a Stratalinker (Stratagene) Free poly-L-lysine molecules on the slides were then blocked with 170 mM succinic anhydride, 3 mM sodium borate in 1-methyl-2-pyrrolidinone (Sigma) for 20 min followed by immersion in boiling water for 2 min. Slides were then rinsed five times in 95% (v/v) ethanol, air-dried, and stored at room temperature.

Fluorescent Sample Preparation

For CGH experiments labelled DNA was generated using a Bioprime labelling kit (BRL Life Technologies) according to the manufacturers instructions, except that 2 μg of DNA was labelled in a 50 μl reaction with 60 μM Cy5-dCTP (or Cy3-dCTP [NEN Boston, Mass.]), plus 60 μM dCTP, and 120 μM dGTP, dATP, dTTP. Total cellular RNA (8 μg) was reverse transcribed overnight at 37° C. with 1200 u Superscript II (BRL, Life Technologies) in 40 μl of reaction buffer supplemented with 500 μM dGTP, dATP and dTTP, 200 μM dCTP, 100 μM DTT, 100 μM Cy5 or Cy3 labelled dCTP and 100× excess random primer (5′-IIINNNNNN-3′, where I is inosine). Labelling reactions were stopped by the addition of EDTA pH8-0 to 90 mM. Cot-1 DNA (25 μg, BRL Life Technologies) was added and unincorporated nucleotides were removed using a Sephadex ProbeQuant G-50 column (Amersham Pharmacia). Samples were heated to 70° C. for 10 min in 80 mM NaOH then neutralised by addition of HCl to 80 mM. 400 μl of 0-5×SSPE (Sigma) was added and the sample was filtered through a 0-1 μM ultra free-MC filter column (Millipore). The volume was then reduced to 30 μl using a Microcon YM-30 filtration unit (Millipore).

Microarray Hybridisation

The microarray grids were denatured by submerging in 70% (v/v) deionised formamide/2×SSC pH7 at 65° C. for 1 min. Slides were then washed sequentially with 70%, 80% and 100% (v/v) ethanol, blow dried with canned air (RS Components) and prewarmed to 37° C. in a hybridisation chamber (BDH Precision Engineers, Cambridge) for 30-60 min. Labelled samples (made from 1-5 μg of genomic DNA, or 2-5 μg of total RNA) in 40 μl of hybridisation mix (0.1% (v/v) Tween 20, 6×SSPE, pH7-4 and 12-5 mM EDTA pH8-0) were heated to 99° C. 2 min, 65° C. 10 min, and 37° C. 10 min, pipetted onto a microarray slide and covered with a Hybrislip (22×60 mm, Sigma). 150 μl of 0-5×SSPE was pipetted underneath the slide, the hybridisation chamber sealed and incubated at 65° C. overnight. The slides were soaked in 2×SSC at 32° C. until the coverslip detached, and then washed with: 2×SSC 32° C. 2 min, 1×SSC/50 mM EDTA pH8-0 r/t 1 min, and 0-05xSSC r/t 1 min. Excess wash solution was removed with canned air.

Image Collection and Analysis

Hybridised microarray slides were scanned in a GenePix 4000A scanner (Axon Instruments). Slides were scanned at PMT (photomultiplier tube) voltage levels that provided an average Cy5:Cy3 hybridisation ratio across the slide of 1-0. Fluorescence ratios (Cy5:Cy3) for individual cDNAs were then determined after subtraction of background using the GenePix Pro 3-0 software (Axon Instruments). For these calculations the hybridisation signal was calculated as the median fluorescence of pixels within the array spot and the background was calculated as the median fluorescence of pixels in a halb surrounding the same array spot.

Immunohistochemistry for HBO1 Protein in Breast Tumour Tissue

Paraffin embedded tissue sections were prepared by washing in Histoclear™ (National Diagnostics) for 5 minutes, followed by 100% ethanol, 70% and 50% ethanol, each for 3 min.

Slides were rinsed for 5 mins with tap water and placed in a microwaveable container, covered with Vector Antigen UnMasking Solution (Vector Laboratories) and heated at 95° C. for 5 mins (microwave).

The container was topped up with water (distilled) and heated at 95° C. for a further 5 mins (microwave), then the slides were allowed to cool in buffer (approx. 60 mins).

After cooling, the slides were washed in distilled water three times for 2 minutes each, followed by methanol/H₂O₂ for 15 mins. After a quick rinse in tap water, the slides were incubated in PBST buffer (1 ml Tween-20 per litre of phosphate buffered saline (10 PBS tablets (BDH) in 1 L deionised water) for 5 mins.

The samples were then incubated in diluted (3 drops to 10 ml PBST) normal blocking serum for 20 mins and then incubated at 4° C. overnight in diluted first antiserum.

The samples were then washed in PBST for 5 mins and incubated for 30 minutes in biotinylated secondary antiserum (3 drops normal blocking serum, 1 drop secondary antiserum to 10 ml PBST)(Vectastain Elite ABC kit, goat IgG, cat.# PK6105, Vector Laboratories).

ABC reagent (Avidin-Biotin Complex, Vector Laboratories) was then made up according to the supplier's instructions (2 drops A and 2 drops B to 5 ml PBST, leave for 30 mins) and incubated with sample for 30 mins. Slides were then washed in PBST for 5 mins and incubated with DAB (3,3′-di-aminobenzidine tetrahydrochloride) substrate (Peroxidase Substrate Kit, DAB, Cat.# SK-4100, Vector Laboratories) (5 ml BDH water+2 drops buffer, mix+4 drops DAB, mix+2 drops H₂O₂, mix) for 10 mins.

After rinsing in water, the samples were then counterstained in haematoxylin for 60 secs, rinsed in tap water, dehydrated, and mounted for analysis.

Results

Microarray Design

A cDNA library was constructed in a plasmid vector using mRNA prepared from the BT474 breast carcinoma cell line, chosen because it contains amplicons on both chromosomes 17 and 20. Conventional CGH analysis using DNA from this line confirmed the presence of these amplicons and demonstrated separate regions of amplification at 17q11-21, 17q22-23 and 20q13. 8,000 cDNA clones were randomly selected from the library and the PCR-amplified cDNA inserts were arrayed onto poly-L-lysine coated glass slides. The rationale behind this approach is that genes overexpressed as a consequence of amplification should be represented within this set of clones. In addition, the random selection of clones from the BT474 library offers the prospect of identifying genes that may not be present on a defined “geneset” array.

Comparative Genomic Hybridisation onto the BT474 cDNA Microarray

CGH studies were carried out on the 8,000 cDNA BT474 breast carcinoma microarray. To detect clones corresponding to sequences transcribed from amplified regions in breast cancer, CGH studies were performed using DNA from the breast carcinoma cell line labelled with Cy5 (red) and DNA from normal muscle labelled with Cy3 (green). The method that we used was similar to that described by Pollack et al. (1999) except that we subjected the cDNA probes attached to the microarray to denaturation in formamide immediately before hybridisation to maximise available sites for binding to genomic DNA sequences. A similar procedure is used to denature the DNA of metaphase chromosomes in conventional CGH analyses (Kallioniemi et al., 1992). We found that inclusion of this step significantly improved the reproducibility and sensitivity of the CGH procedure.

To establish the normal distribution of the hybridisation ratios a control CGH experiment was performed in which the microarray was hybridised simultaneously to normal muscle DNA labelled with Cy5 (red) and to a sample of the same muscle DNA labelled with Cy3 (green). A much greater variation in hybridisation ratios was observed in a parallel BT474:muscle CGH experiments and using a statistical outlier test 231 clones were identified with red:green hybridisation ratios greater than those observed in the muscle:muscle CGH study.

A potential problem with this microarray design is that it would be expected to contain a significant proportion of cDNA derived from mitochondrial transcripts. The position of mitochondrial clones was determined by hybridising the microarray to fluorescently labelled human mitochondrial DNA. Overall 11% of the sequences were mitochondrial and these array spots were removed electronically prior to analysis of information from the microarray.

Identification of Amplified Genes on Chromosome Arm 20q and 17q

DNA sequencing and database searches of amplified genes detected by CGH onto the BT474 cDNA microarray identified 39 clones, representing 20 distinct genes, on chromosome arm 20q.

CGH analyses onto the BT474 cDNA microarray identified 142 clones representing 30 distinct genes that mapped to chromosome arm 17q. Representative genes were used as probes on Southern blots to confirm the presence of amplification in the BT474 cell line. Those 17q genes for which the precise positions on the 3000 cRay radiation-hybrid map were available, clustered in two main groups at 302-316 cRay and at 338-383 cRay, which correspond respectively to the 17q11-21 and 17q22-23 amplicons.

Expression Studies.

To assess whether amplification was associated with high levels of expression, we carried out parallel expression studies on BT474 cDNA microarrays using cDNA from the mammary epithelial cell line HB4a as a reference. HB4a cells exhibit many of the characteristics of normal mammary luminal epithelial cells and can be transformed in vitro by expression of high levels of ERBB2 (Harris et al, 1999). BT474 cDNA microarrays were co-hybridised with Cy3 labelled HB4a cDNA, plus Cy5 labelled cDNA from either the BT474 or the SK-BR-3 breast cancer cell lines. The BT474 cell line contains all three amplicons and the SK-BR-3 line contains amplicons at 20q13 and 17q11-21. To check the effectiveness of this approach for detecting increased expression, we used 12 amplified genes showing hybridisation ratios (BT474:HB4a) of at least 2.8-fold as probes on northern blots of BT474 and HB4a RNA. All 12 were confirmed to have at least the same level of over-expression that was detected in microarray based expression studies.

In addition, for the BT474 cell line, patterns of gene expression along chromosome 17 were plotted using data obtained from duplicate ICR-Geneset hybridisations. Notably two main loci of over-expressed genes were observed which correspond to the amplicons and 17q11-12 and 17q22-23 identified in the CGH experiments.

The microarray expression data was used as a guide for selecting potentially over-expressed genes. To further assess the importance of the genes selected, we carried out northern analyses on RNA from nine breast cancer cell lines. In addition, to be absolutely sure that any increased expression was associated with DNA amplification, parallel Southern bolt studies were also performed.

Within the 17q22-23 amplicon the HBO1 gene has higher levels of expression in the BT474 and MDA-MB361 lines, the only two cell lines in our study that contained this amplicon. Higher levels of HBO1 transcripts were also observed in the absence of amplification in the breast cancer carcinoma line ZR-75-1.

Immunohistochemistry for HBO1 protein in breast tumour tissue HBO1 expression in paraffin embedded breast tumour sections was determined using immunohistochemistry.

Immunohistochemistry for HBO1 was conducted following antigen retrieval in Vector antigen unmasking solution and microwave treatment at 95° C. for 10 minutes using ABC reagents (Vector Laboratories), following a standard protocol as described above. The primary antibodies used were HBO1 (T20), HBO1 (N18) and a comparable goat IgG control (Santa Cruz) at 0.8 μg/ml IgG. Peroxidase staining was then performed using 3,3′-di-aminobenzidine tetrahydrochloride (DAB) substrates and samples were subsequently counterstained with haematoxylin and visualised using a light microscope at 4× and 10× magnification.

At the concentration of antibody used, the staining intensity using the HBO1 antibodies was greater than that observed for the IgG control, indicating that the staining was specific for HBO1. HBO1 expression appeared to be more pronounced in areas of tumour compared to normal tissue and stroma.

The distinct feature of our study is that the arrayed clones were randomly selected from a cDNA library prepared from a tumour cell line containing the amplicon under investigation. The method relied on (i) sequences that were overexpressed as a consequence of amplification being present in the selected set of cDNAs and (ii) clones derived specifically from the amplified regions being highlighted in the CGH analyses. The advantage of this technique is that genes do not have to be preselected in order to be detected. This approach has proven remarkably successful, leading to the identification of 50 amplified and expressed genes within the 17q11-21, 17q22-23 and 20q13 amplicons. Hybridisation of cDNA targets prepared from breast cancer cell lines was used to assess the levels of expression of amplified genes relative to those present in an immortal mammary luminal epithelial cell line.

Amplicons frequently span many genes and the importance of individual genes within the amplicon is often debated. The amplicons on 17q and 20q in breast cancer are no exception. Tanner et al. (1996) and Pinkel et al. (1998) have, for example, provided evidence that there are at least three separate regions of amplification at 20q13 and in the present study we found that amplified genes are linked in four groups, perhaps suggesting an even higher level of complexity.

The HBO1 histone acetyl transferase gene (Izuka & Stillman, 1999) is shown by our studies to be a candidate oncogene for involvement in the amplicon at 17q22-23. The gene was expressed at higher levels in the ZR-75-1 breast cancer cell line in the absence of amplification.

### REFERENCES

Clark J et al. (1994). Nat. Genet., 7, 502-508.
Collins C et al (1998). Proc. Natl. Acad. Sci. USA, 95, 8703-8708.
Cordon-Cardo C et al (1994). Cancer Res., 54, 794-799.
Couch F J et al (1999). Cancer Res., 59, 1408-1411.
Fukumoto M, et al (1988). Proc. Natl. Acad. Sci. USA, 85, 6846-6850.
Gayther S A et al (2000). Nat. Genet., 24, 300-303.
Gracia E et al. (1996). Hum. Mol. Genet., 5, 595-600.
Harris R A et al (1999). Int J. Cancer, 3, 477-484.
Izuka M and Stillman B. (1999). J. Biol. Chem., 274, 23027-23034.
Kallioniemi A, et al (1992). Science, 258, 818-821.
Kallioniemi O P et al (1993). Trends Genet., 10, 352-357.
Marks P A, et al (2000). J. Natl. Cancer. Inst., 92, 1210-6.
Pinkel D et al. (1998). Nat. Genet., 20, 207-211.
Pollack J R, et al (1999). Nat. Genet., 23, 41-46,
Sambrook J et al (1989). Cold Spring Harbor Laboratory Press.
Schwab M & Aml 1C. (1990). Genes Chrom. & Cancer, 1, 181-193.
Schwab M. (1998). BioEssays, 20, 473-479.
Tanner M M et al (1996). Cancer Res., 56, 3441-3445.
Tomasetto C et al (1995). Genomics, 28, 367-376.
Wilkinson M. (1988). Nucl. Acid. Res., 16, 10394.
Wu G-J et al (2000). Cancer Res., 60, 5371-5375.

1. A method of diagnosis or prognosis of breast cancer, which method comprises:
providing a sample of breast tissue which is, or is suspected to be, malignant from a patient;
determining the level of HBO1 expression or amplification in said sample; and
comparing said level with a control to determine whether the level of HBO1 expression is abnormal.

2. A method according to claim 2 wherein said control is normal breast tissue from said patient.

3. A method according to claim 1 or claim 2 wherein the level of HBO1 expression or amplification is determined by measuring the level of nucleic acid sequence encoding said HBO1 polypeptide.

4. A method according to claim 3 comprising contacting the sample with a nucleic acid probe which specifically hybridises to said nucleic acid sequence,
determining the level of binding of the probe to the sample; and,
comparing said level with a control to determine whether the level of HBO1 expression is abnormal.

5. A method according to claim 1 or claim 2 wherein the level of HBO1 expression or amplification is determined by measuring the level of said HBO1 polypeptide.

6. A method according to claim 5 comprising contacting the sample with a specific binding member directed against an HBO1 polypeptide,
determining the level of binding of the specific binding member to the sample; and,
comparing said level with a control to determine whether the level of HBO1 expression is abnormal.

7. A method of identifying and/or obtaining a compound as a putative anti-cancer agent, the method comprising;
providing HBO1 protein and a substrate therefor, under conditions where, in the absence of inhibitor, the protein is able to acetylate the substrate;
bringing a putative inhibitor into contact with said protein and substrate to determine if the putative inhibitor alters the ability of the HBO1 protein to acetylate the substrate; and
selecting a putative inhibitor which alters the ability of the HBO1 protein as an inhibitor of breast cancer cell growth.

8. A method according to claim 7 wherein the substrate is a histone molecule.

9. A method according to claim 7 comprising the step of determining the ability of the inhibitor to inhibit the growth of breast cancer cells in vitro.

10. A method according to claim 7 comprising identifying the test compound as a putative anti-cancer agent.

11. A method according to claim 10 comprising isolating and/or purifying the test compound.

12. A method according to claim 11 comprising modifying the test compound to optimise its pharmaceutical properties.

13. A method according to claim 7 comprising formulating said test compound with a pharmaceutically acceptable excipient.

14. A compound identified as a putative anti-cancer agent by a method of claim 10.

15. A pharmaceutical composition comprising the compound of claim 14 and a pharmaceutically acceptable excipient.

16. A compound according to claim 14 for use in a method of treatment.

17. Use of a compound according to claim 14 in the manufacture of a medicament for use in the treatment of breast cancer.

18. A nucleic acid encoding HBO1 or its complement or a fragment thereof for use in a method of treatment.

19. A nucleic acid according to claim 18 wherein said method is for the treatment of breast cancer.

20. Use of nucleic acid encoding HBO1 or its complement or a fragment thereof in the manufacture of a medicament for the treatment of breast cancer.

21. A method of treating a breast cancer condition in an individual, the method comprising reducing the acetylase activity of HBO1 polypeptide in one or more cells of said individual.

22. A method according to claim 21 wherein the activity of HBO1 polypeptide is reduced by administering an antagonist of HBO1 to said individual.

23. A method according to claim 21 wherein the activity of HBO1 polypeptide is reduced by decreasing or abolishing expression of the HBO1 polypeptide.

24. A method according to claim 23 wherein expression of the HBO1 polypeptide is abolished or reducing by administering a nucleic acid according to claim 18.


SEQUENCE LISTING (4 sequences)

SEQ ID NO: 1 - DNA, 18 bp, Artificial Sequence
  Description of Artificial Sequence Primer
gtaaaacgacggccagtg

SEQ ID NO: 2 - DNA, 19 bp, Artificial Sequence
  Description of Artificial Sequence Primer
ggaaacagctatgaccatg

SEQ ID NO: 3 - DNA, 23 bp, Artificial Sequence
  Description of Artificial Sequence Primer
gagcggataacaatttcacacag

SEQ ID NO: 4 - DNA, 18 bp, Artificial Sequence
  Description of Artificial Sequence Primer
gttttcccagtcacgacg